(19) United States
(12) Patent Application Publication
(10) Pub. No.: US 2026/0342035 A1
(43) Pub. Date: Oct. 29, 2026

(54) PULSATING LASER AND METHOD OF CONTROL THEREOF

(71) Applicant: LASER TEAM MEDICAL LTD., Katzrin (IL)

(72) Inventors: Alon SHACHAM, Katzir (IL); Salman NOACH, Jerusalem (IL); Rotem NAHEAR, Kiryat Arba (IL); Neria SULIMAN, Jerusalem (IL)

(73) Assignee: LASER TEAM MEDICAL LTD., Katzrin (IL)

(21) Appl. No.: 19/107,325

(22) PCT Filed: Aug. 29, 2023
(86) PCT No.: PCT/IL2023/050915

§ 371 Date: Feb. 27, 2025

Related U.S. Application Data
(60) Provisional application No. 63/402,128, filed on Aug. 30, 2022.

(51) Int. Cl. H01S 3/113; A61B 17/00; A61B 18/00; A61B 18/20; H01S 3/10; H01S 3/117

(52) U.S. Cl. CPC H01S 3/113; A61B 18/20; H01S 3/10069; H01S 3/117; A61B 2017/00199; A61B 2018/00577; A61B 2018/00589; A61B 2018/205547; H01S 2302/00

(57) ABSTRACT

Discloses is a nanosecond scale (2-150 ns) pulsed laser system with wavelength between 1900 to 2020 nm, comprising: a laser cavity; a gain medium disposed within the laser cavity; a pump configured to optically pump a lasing medium; and optionally, a q-switching element positioned within the laser cavity. The system may further include a controller, configured to: receive, from a user interface, an input comprising a desired ablation depth and desired coagulation diameter of a laser treatment; determine a required number of pulses to be provided at a location on the tissue based on the pulse's energy and the desired ablation depth, determine pulses frequency based on the pulse's energy, the desired coagulation diameter, and control the laser system to produce the laser pulses.

### CROSS-REFERENCE TO RELATED APPLICATIONS

This application claims the benefit of priority of U.S. Patent Application No. 63/402,128, filed Aug. 30, 2022, which is hereby incorporated by reference in its entirety.

### FIELD OF THE INVENTION

Exemplary embodiments of the present invention relates generally to pulsating lasers. More specifically, the present invention relates to controlling pulsating lasers to achieve a desired ablation depth and desired coagulation diameter.

### BACKGROUND OF THE INVENTION

Laser systems are widely used in medical fields, for example, to perform precise surgeries or any dermal intervention. The benefits of laser systems are their capabilities of producing a laser beam with a high energy output focused to miniscule, precise location. Current laser systems used in the art may include active or passive Q-switchers, which are used to create laser pulse at the nano second scale.

In certain medical procedures, for example, laser coagulation, a laser is used to create a small ablation in the retina of the eye. The ablation must be as precise as possible, in order to prevent growth of abnormal blood vessels or tears in the retina from damaging the retina. In many cases, a required ablation well can be determined to prevent further retina damage, where the ablation well has a required depth and diameter (or “width”) in which the laser must produce. Additionally, the width of the thermal damage around the ablation well, the coagulated tissue, is important to the procedure's success.

Accordingly, there is a need for a laser system capable of producing a controlled ablation well and control the surrounding coagulation in tissue.

### SUMMARY OF THE INVENTION

Embodiments of the present invention are directed to a nanosecond scale (2-150 ns) pulsed laser system with wavelength between 1900 to 2020 nm, comprising: a laser cavity; a gain medium disposed within the laser cavity; a pump configured to optically pump a lasing medium; and optionally, a q-switching element positioned within the laser cavity. The system may further include a controller, configured to: receive, from a user interface, an input comprising a desired ablation depth and desired coagulation diameter of a laser treatment; determine a required number of pulses to be provided at a location on the tissue based on the pulse's energy and the desired ablation depth, determine pulses frequency based on the pulse's energy, the desired coagulation diameter, and control the laser system to produce the laser pulses.

Some aspects of the present invention are directed to a method for controlling a laser system, the method comprising: receiving, from a user interface, an input comprising a desired ablation depth and desired coagulation diameter of a laser treatment; determining a required number of pulses to be provided at a location on the tissue based on the pulse's energy and the desired ablation depth, determining pulses frequency based on the pulse's energy, the desired coagulation diameter and controlling the laser system to produce the laser pulses.

In some embodiments, the controller is configured to determine the frequency and the required number of pulses based on data stored in a storage system associated with the controller. In some embodiments, the data is stored in a lookup table. In some embodiments, the required number of pulses is determined based on the desired depth of the ablation. In some embodiments, the required frequency is determined based on the desired diameter of the ablation and/or coagulation.

In some embodiments, the q-switching element is selected from an active q-switching element and a passive q-switching element.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of an exemplary laser system, according to some embodiments of the invention;

FIG. 2 is a schematic illustration of another exemplary laser system, according to some embodiments of the invention;

FIG. 3 is a block diagram of another laser system according to some embodiments of the invention;

FIG. 4 is a block diagram of a computing device for a laser system according to some embodiments of the invention; and

FIG. 5 is a flowchart of a method of controlling a laser system according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Disclosed herein is a laser system, e.g. a laser operating in the 2 micrometers (“μm”) wavelength range, for example, 1.8-2.2 μm, 1.85-2.2 μm, 1.85-2.05 μm, 1.85-2.0 μm, 1.9-2.1 μm, or any value in between. In some embodiments, The laser is a pulsed laser, which may be used for a variety of applications, e.g. surgery, for any type of tissue ablation and/or coagulation, military applications, material processing, optical communication, LIDAR or the like.

In some embodiments, the laser system disclosed herein may comprise a q-switching element to control a laser beam of the laser system. As used herein the term “q-switching element” may refer to either a passive q-switch (also referred to as “saturable absorber (SA)”) or an active q-switch. In some embodiments, a q-switching element may control the laser beam, for example, to modulate a pulse of the laser beam. In a non-limiting example, a q-switching element may be comprised of a saturable absorber (SA), which may passively absorb a laser beam in order to produce a pulsed laser beam at a desired energy, further discussed herein below.

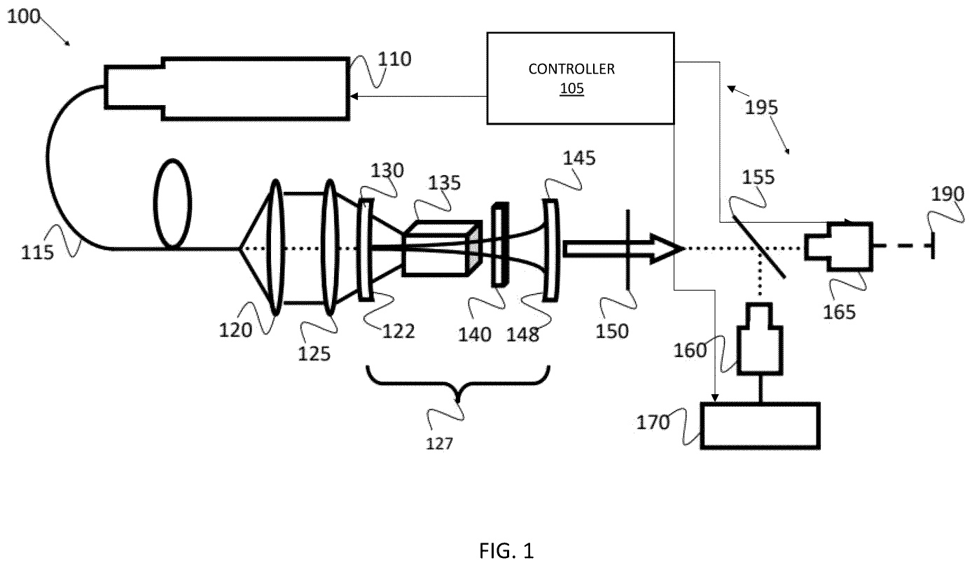

Reference is now made to FIG. 1 detailing a schematic illustration of a laser system, according to some embodiments of the invention. According to some aspects, a laser system 100 may comprise a pump 110 (such as pump diode) configured to optically pump a lasing medium. In some embodiments, the pump 110 may be optically coupled into a fiber 115 to generate a gain for a laser. In some embodiments, pump diode 110 may be operated in a continuous wave mode or quasi-continuous wave mode.

In some embodiments, pump 110 may be tuned to provide a beam having a wavelength which matches the corresponding absorption peaks of a gain medium as described below. There are various pump schemes and pumping configurations well known in the art and some of them may be applied to the present disclosure application. In some embodiments, pump diode 110 may include direct pumping, the pump diode may deliver into fiber 115. In some embodiments, the pump diode 110 configurations may include a side pump and an end pump.

In some embodiments, laser system 100 may further include a gain medium 135. In some embodiments, the gain medium 135 may be disposed within a laser cavity 127. Non-limiting exemplary gain media 135 are selected from materials (also referred to as “laser crystals”) doped with a rare-earth element. In some embodiments, the material is a crystal selected from: Yttrium Aluminum Garnet (“YAG”), and Yttrium Lithium Fluoride (“YLF”), and Yttrium Aluminum Phosphorus (“YAP”). In some embodiments, the rare earth element is selected from Thulium (Tm), Holmium (Ho), Erbium (Er), or any combination thereof.

Further non-limiting exemplary gain media 135 are selected from: Tm:YAG, Tm:YVO₄, Tm:YLF, Tm:YAP or Tm:LuAG. In some embodiments, the concentration of the Tm³⁺ dopant in the host crystal material of the laser crystal is inversely proportional to the length of the laser crystal. In some embodiments, the concentration of Tm³⁺ dopant is between about 0.2 wt. % to about 8 wt. % and any value in between, for example, 1-6 wt. %, 2-7 wt. %, 2-8, 0.5 to 7 wt. % and the like.

In some embodiments, laser system 100 may comprise a first optical element 130 and a second optical element 145. In some embodiments, laser system 100 may comprise a first collimation lens 120 and a second focusing lens 125. In some embodiments, first optical element 130 may be selected from a lens, a reflector, a mirror, e.g., a convex mirror, and a prism. In some embodiments, the radius of curvature of the concaveconvex mirror may be in the −50 to plano and 30 to plano respectively and any value in between, for example, −100 to plano and 50 to plano, −200 to plano and 100 to plano and the like. In some embodiments, the first optical element 130 may be positioned in a light-path e.g., approximately along a longitudinal axis 190 of the laser system 100.

In some embodiments, laser system 100 may comprise a q-switching element 140 e.g., saturable absorbing passive Q-switches. In some embodiments, the q-switching element 140 may be in the form of a thin layer or film. In some embodiments, non-limiting exemplary q-switching element 140 may comprise a material selected from doped ZnS crystals, and doped ZnSe crystals e.g., chromium doped ZnSe crystals, chromium doped ZnS crystals, Ho doped materials, such as, Ho:YAG, Ho:YLF, Ho:YAP Ho doped fiber or a combination thereof. In some embodiments, further non-limiting exemplary q-switching element 140 may comprise a material selected from doped silver halide or a chalcogenide.

In some cases, the Cr:ZnSe and the Chromium doped Zinc Sulfide (“Cr:ZnS”) SA may have a relatively high absorption cross-sections, thus not requiring a focusing mode to a small area on the SA. This may provide more flexibility with respect of the resonator. Optionally, the Cr:ZnSe and the Cr:ZnS saturable absorbers are capable of a low saturable intensity, which may lead to reduced risk of damage during Q-switched operation.

In some exemplary embodiments of the subject matter, the Cr:ZnS crystal saturable absorber may be applied in several passive Q-switch (“PQS”) lasers, e.g. Ho:YAG, Tm:KY(WO₄), Tm:KLu(WO₄), or the like. In some cases, the SA may fulfill a passive Q-switch when σ_{SA}/A_{SA}>σ_{g}/A_{g}, where σ_{SA} and σ_{g} represent the absorption cross section of the saturable absorber and the emissions cross section of a gain medium at the lasing wavelength, respectively, and A_{SA} and A_{g} may be the mode area at the saturable absorber and gain medium.

In some embodiments, laser system 100 may comprise gain (lasing) medium 135, and q-switching element 140, which are disposed along longitudinal axis 190. In some embodiments, horizontal axis 190 may be defined as up to ±45 degrees in from the longitudinal axis 190.

In some embodiments, first optical element 130 may be located at a proximal first end of laser cavity 127. In some embodiments, first optical element 130 may include a first surface 120 and a second surface 125. In some embodiments, the first surface 122 may be directed substantially towards the laser cavity 127 and gain medium 135. In some embodiments, the first optical element 130 may be a high reflecting mirror, as is well known in the art. In some embodiments, first surface 122 may be coated with silver, a dielectric, or some similar coating to provide the high reflective properties. In some embodiments, first surface 122 may be characterized as High Transmission (“HT”) of the beam received from pump diode 110. In some embodiments, first surface 122 may be characterized as having High Reflection (“HR”) of the wavelength in the infra-red (IR) range, e.g., 1800-2100 nm, 1800-2000 nm, 2000-2100 nm and any range and value herein between.

In some embodiments, first optical element 130 may be configured to be a diverging optical element; either as a reflecting convex surface, as a plano element, or as a plano-concave optical element. In some embodiments, the light striking first optical element 130 may diverge as it reflects back toward gain medium 135. In some cavities, dependent upon gain medium 135, it may be beneficial to place an aperture (not illustrated) adjacent to first optical element 130 so as to prevent high divergent light from reentering gain (lasing) medium 135, e.g., due to waveguiding effects. In some cases, the first optical element 130 may be a lens or birefringent plate, which may enable tunability of the gain medium 135.

In some embodiments, the first optical element 130 may be collimated by first surface 122 and made available to again seed gain medium 135 for further amplification while retaining the low order mode quality originally established.

In some embodiments, laser system 100 may have a second optical element 145, also referred to as output mirror. In some embodiments, second optical element 145 may be positioned at a proximal second end of laser cavity 127. In some embodiments, second optical element 145 may be selected from a lens, a reflector, a mirror and a prism. In some embodiments, second optical element 145 may be positioned in a light-path of the beam e.g. proximately along the longitudinal axis 190 of laser system 100.

In some embodiments, second optical element 145, also known as the output mirror, may be positioned at second end of laser cavity 127, opposite to first optical element 130. In some embodiments, second optical element 145 may act as a regenerative and/or as transmissive interface for laser beam exiting laser cavity 127. Optionally, the length of the laser cavity 127 may be within a range of 30-700 nm and any range in between, for example, 100-410 mm, 50-500 nm, 90-450 nm, 125-400 nm and the like. In some embodiments, second optical element 145 may have a second surface 148. In some embodiments, second surface 148 may be coated for partial reflectivity, dependent upon the gain of gain medium 135. In some embodiments, second surface 148 may be antireflection coated for the light being amplified.

In certain embodiments, laser system 100 may be monitored via a monitoring system 195. In some embodiments, monitoring system 195 may include a controller 105 configured to receive an input signal from one or more components of system 100 and to control at least pump 110 based on the received signals. In some embodiments, monitoring system 195 may provide a manner for monitoring and obtaining experimental data from the laser system 100 according to its output.

In some embodiments, the monitoring system 195 may comprise an optical filter 150. In some embodiments, optical filter 150 may be optically connected to laser cavity 127 at least partially by free space light propagation. In some embodiments, a wavelength of a beam emitting through the optical filter 150 may be tuned, for example, by altering the angle of the optical filter 150 with respect to the incident optical beam inside the laser cavity 127. In some embodiments, optical filter 150 may comprise one or more arrayed waveguide gratings. In some embodiments, controller 105 may control optical filter 150.

In some embodiments, monitoring system 195 may comprise a beam splitter 155. In some embodiments, beam splitter 155 may have a predetermined power ratio between reflected and transferred components of a laser beam that incident with the original laser beam. In some embodiments, beam splitter 155 may be insensitive to the direction of the polarization of the incident laser beam and its reflected and transferred components thus there may be no requirements for the incident beam's polarization. In some embodiments, the angle of the beam splitter 155 in relation to the incident beam may be, for example, in a 45-degree angle. In some embodiments, exemplary range of angles for beam splitter 155 may be: 0-60 degree.

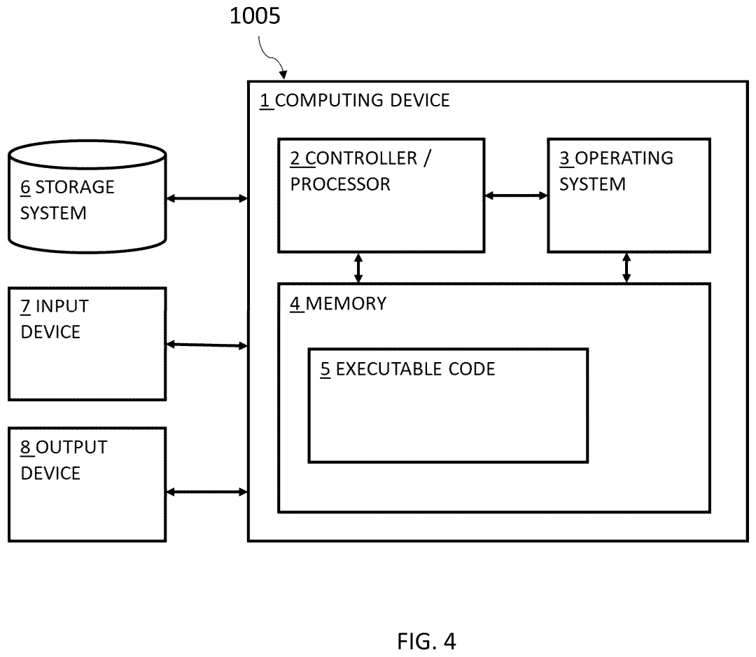

In some embodiments, monitoring system 195 may comprise an oscilloscope 170. In some embodiments, oscilloscope 170 may allow displaying the intensities of the beams emitting optical filter 130. In some embodiments, oscilloscope 170 may be operatively connected to a photodiode 160. In some embodiments, photodiode 160 may allow converting a component of a laser beam to an electric signal. In some embodiments, the monitoring system 195 may have a power (or energy) meter 165. In some embodiments, power meter 165 may allow measuring the power level of a laser source beam. In some embodiments, power meter 165 may have a sensor and/or a photodetector and a variable electrically connected. In some embodiments, controller 105 may control power meter 165 based on signals received from the sensor and/or the photodetector or based on information received from a storage system such as storage system 6 discussed with respect to FIG. 4.

In some exemplary embodiments of the subject matter, the laser diode 110 provides a beam through an optical fiber with certain properties, e.g., a 105 μm core diameter and a numerical aperture (NA) of 0.22, and emitting within a power range of 6-30 W at approximately a wavelength of 793 nm. In some embodiments, a laser beam may collimate and focus into an initial pump spot of 200-350 μm diameter on the gain medium 135 via the first optical element 130. Optionally, the gain medium 135 has a length of 10 mm and a cross-section of 3×3 mm. In some embodiments, the Tm-doped concentration may be at approximately 2.5%-4%. Optionally, the gain medium 135 may be wrapped in Indium foil and placed in a copper holder (not shown), where the copper holder is inserted into a circulating water-cooled aluminum housing or connected to thermo-electric cooling.to maintained temperature at approximately 18-25 °C.

In some embodiments, the q-switching element 140 may be positioned to provide a maximized energy pulse without damaging the q-switching element 140 surface, e.g., approximately at 8 cm from an output of the pump diode 110 or fiber 115. Optionally, the q-switching element 140 may be 2 mm thick with apertures of 4×4 mm, also placed in a copper holder. In some embodiments the q-switching element 140 may be uncoated and position in Brewster angle to increase the energy pulse without damaging the q-switching element 140 surface.

In some embodiments, laser system 100 may have a housing. In some embodiments, the housing may be made of a rigid, durable material, such as, without limitation, aluminum, stainless steel, a hard polymer and/or the like. In some embodiments, the housing may have a cylindrical, conical, rectangular or any other suitable shape. In some embodiments, the housing may prevent unwanted foreign elements from entering thereto.

In some exemplary embodiments of the subject matter, the distance between the first optical element 130 to the gain medium 135 may be within a range of 10-20 mm. In some embodiments, the distance between the gain medium 135 and the q-switching element 140 may be within a range of 85-160 mm. In some embodiments, the distance between the q-switching element 140 and the second optical element 145 may be within a range of 80-105 mm.

In some embodiments, a ratio of an area of the laser beam within q-switching element 140 (e.g., the saturable-absorber) to an area of the beam area within the gain medium is more than 1.3. In some non-limiting examples, the gain medium 135 may comprise a spot radius within a range of 128-150 μm, and the q-switching element 140 (e.g., a SA) may comprise a spot radius within a range of 410-785 μm. In some embodiments, the ratio of an area of the laser beam within the saturable-absorber to an area of the beam area within the gain medium is more than 3.5. In some embodiments, a ratio between the saturable-absorber radius spot on the saturable-absorber and a gain medium radius spot on the gain medium is within a range of 1.7-7.

One specific exemplary embodiment of the subject matter, a distance between the first optical element 130 to the gain medium 135 may be 10 mm. In some embodiments, the distance between the gain medium 135 and the q-switching element 140 may be 85 mm. In some embodiments, the distance between the q-switching element 140 and the second optical element 145 may be 105 mm. In some embodiments, the gain medium 135 may comprise a spot radius within a range of 115 μm, and the q-switching element 140 may comprise a spot radius within a range of 450 μm. In some embodiments, the total length of the laser cavity 127 may be 215 mm. In some embodiments, the ratio between a q-switching element spot radius and a gain medium spot radius may be, for example, 3.46.

In some embodiments, controller 105 may further be configured to: receive an input containing a desired depth and diameter of a laser ablation from a user interface, determine a frequency of a laser beam based on the desired diameter, determine a lasing power and the number of pulses per ablation well of the laser beam based on the desired depth, and control the laser system (e.g., laser system 100) to produce the laser beam. In some embodiments, controller 105 may receive a signal, containing a desired depth and diameter of a laser ablation, from an external device associated with laser system 100. In some embodiments, controller 105 may control a frequency and lasing power of laser system 100 to produce a desired ablation depth and diameter according to the received input. In some embodiments, a selected operating frequency and lasing power may be determined from a reference table, discussed herein below under “Experimental Results”. In some embodiments, controller 105 may control pump 110 to produce a laser beam at a determined lasing power and frequency according to the received input. In some embodiments, controller 105 may control an active q-switching element 140 to produce a pulsed laser beam at a determined frequency according to the received input. In some embodiments the controller 105 control a scanning element. In some embodiment the controller 105 control the pulse frequency with the scanning elements, deliver to the same ablation well only fraction of the laser system 100 pulses. In some embodiment the controller 105 control the scanning element to create some ablation well at the same time, deliver each pulse to the next well, and repeating the pattern at the desired frequency.

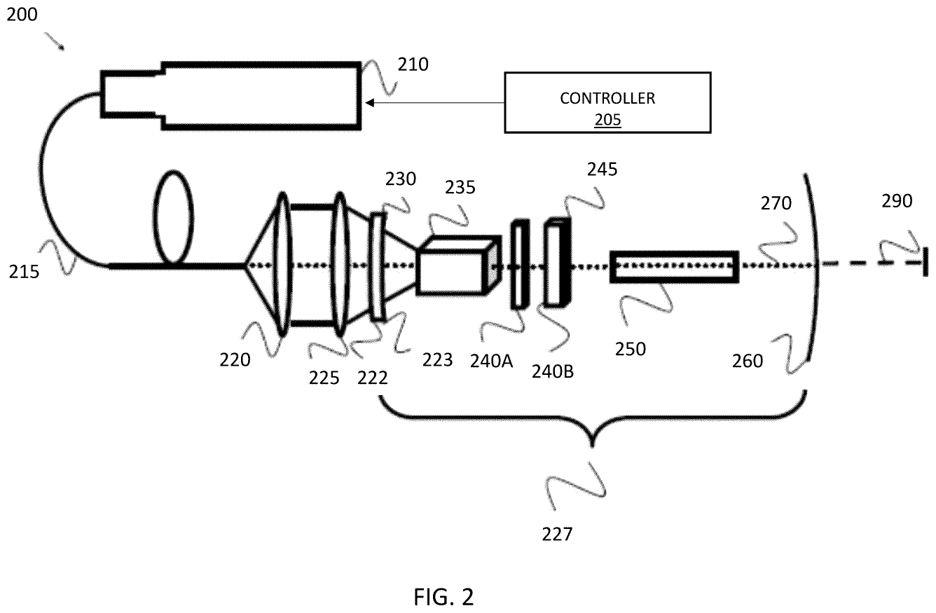

Reference is now made to FIG. 2 detailing a schematic illustration of an exemplary laser system according to some embodiments of the invention. In some embodiments, a laser system 200 may comprise substantially the same components, elements, and units as laser system 100 discussed hereinabove. In some embodiments, laser system 200 may comprise a pump 210 configured to optically pump a lasing medium. In some embodiments, pump 210 may be optically coupled into a fiber 215 to generate a gain for a laser. In some embodiments, pump 210 may be operated in a continuous wave mode or quasi-continuous wave or pulsed mode.

In some embodiments, laser system 200 may comprise a gain medium 235 and q-switching element 250, wherein gain medium 235 comprises substantially the same aspects, embodiments, and capabilities as gain medium 135 of laser system 100 illustrated and discussed hereinabove with respect to FIG. 1. In some embodiments, q-switching element 250 may be an active q-switching element or a passive q-switching element. As discussed herein below.

In some embodiments, system 200 may further include a controller 205, for controlling at least some of the parameters of laser pulse, as discussed herein below.

In some embodiments, laser system 200 may comprise a first optical element 230 (e.g., an input mirror). Optionally, the laser system 200 may comprise one or more lenses, which may allow to optically couple the pump source 210 to gain medium 235. Optionally such lenses may focus a beam emerging from the pump source 210, allowing a minimum spot size inside the gain medium 235 (e.g., 100 to 500 μm). Optionally, laser system 200 may comprise a first collimation lens 220 and a second focusing lens 225.

In some embodiments, optical element 230 may be selected from a lens, a reflector, a mirror, e.g., a convex mirror, and a prism. In some embodiments, optical element 230 may be positioned in a light-path of a laser beam e.g., approximately along a longitudinal axis 290 of laser system 200. In some embodiments, one or more from: optical element 230, first collimation lens 220, and a second focusing lens 225 may allow pump source 210 to be optically coupled to the gain medium 235. Optionally, optical element 230, first collimation lens 220, and second focusing lens 225 may be positioned in a light-path of a laser beam.

Optionally, input mirror 230 may be located at the first end of laser cavity 227. Optionally, input mirror 230 may be configured to serve as a diverging optical element; either as a reflecting convex surface, as a plano element, or as a plano-concave optical element. In some embodiments, the light striking the input mirror may diverge as it reflects back toward the gain medium. In some cavities, dependent upon gain medium, it may be beneficial to place an aperture adjacent to the input mirror so as to prevent high divergent light from reentering the gain (lasing) medium, e.g., due to waveguiding effects.

Optionally, input mirror 230 may include a first surface 222 and a second surface 223. In some embodiments, second surface 223 may be directed substantially towards the laser cavity 227 and gain medium 235. Optionally, second surface 223 may be coated with silver, a dielectric, or some similar coating to provide the high reflective properties e.g., in order to serve as input mirror. In some embodiments, first surface 222 may be characterized as High Transmission (“HT”) of the beam received from pump source 210. Optionally, surface 223 may be characterized as having high reflection (“HR”) of the wavelength in the infra-red (IR) range, e.g., 1500-3500 nm, e.g., 1800-2200 nm. Optionally, surface 223 may be characterized as having a HT to the wavelength of the pump source 210 (e.g., 700-800 nm).

In some embodiments, laser system 200 may have one or more etalons (e.g., two) 240A and 240B, which are positioned a light-path of the laser beam. Optionally, the second etalon 240B is positioned next to the first etalon 240A, so as the first and the second etalons are positioned a light-path of the laser beam.

Optionally, the etalons 240A and 240B are positioned along a horizontal axis 290 comprising gain medium 235. In some embodiments, horizontal axis 290 may be defined as up to +60 degrees from a longitudinal axis.

Optionally, etalons 240A and 240B, provide a tunable spectral range and a narrow spectral bandwidth of the laser. Optionally, the transmission wavelength band of the laser light is dictated by reflectivity, a thickness, and a refractive index of etalons 240A and 240B, and thus a pulse width thereof is adjusted. Optionally, the tunability range is at least 10 nm, at least 14 nm, at least 20 nm, at least 25 nm, at least 30 nm, at least 35 nm. Optionally, the tunability range is from 8 to 50 nm, or, in some embodiments from 8 to 15 nm, or, in some embodiments from 10 to 15 nm, or, in some embodiments from 15 to 20 nm, or, in some embodiments from 20 to 30 nm, or, in some embodiments from 30 to 35 nm, or, in some embodiments from 35 to 40 nm.

In some embodiments, the tunability range may depend on the gain medium 235. Optionally, the tunability range may depend on reflectance degree of the output coupler, and/or transmission degree of the q-switching element.

Optionally, etalon 240A is thinner than etalon 240B. Optionally, etalon 240A has a thickness of 1 to 100 μm, or, in some embodiments, from 10 to 40 μm, or, in some embodiments, from 20 to 30 μm or, in some embodiments, from 30 to 40 μm or, in some embodiments, from 40 to 60 μm or, in some embodiments, from 60 to 100 μm. In some embodiments, etalon 240A has a thickness of 5, 10, 15, 20, 25, 30, 35, 40, 45, or 50 μm, including any value and range therebetween.

Optionally, etalon 240B has a thickness of 100 to 600 μm, or, in some embodiments, from 200 to 600 μm, or, in some embodiments, from 300 to 600 μm. Optionally, etalon 240B has a thickness of 100, 150, 200, 250, 300, 350, 400, 450, 500, 550, or 600 μm, including any value and range therebetween.

Optionally, a thickness ratio of etalon 240A to etalon 240B is from 1:5 to 1:40, respectively. Optionally, a thickness ratio of etalon 240A to etalon 240B is 1:5, 1:10, 1:15, 1:20, 1:25, 1:30, 1:35, or 1:40, respectively, including any value and range therebetween.

Without being bound by any particular theory or mechanism, it is assumed that the thinner etalon allows tunability of the spectral range. In some embodiments, a thinner etalon thickness provides a wider tunability of the spectral range. Further, and without being bound by any particular theory or mechanism, it is assumed that the thicker etalon response for the spectral bandwidth narrowing, with the maximum thickness being limited to avoid the occurrence of two spatial adjacent modes. In some embodiments, the use of two etalons provides both features of spectral range tunability and spectral bandwidth narrowing.

Optionally, etalon 240A or etalon 240B may comprise a full or a partial reflecting material e.g., in the form of a coating 245.

Optionally, laser system 200 may have a Q-switching element 250, which may allow to operate the laser system 200 in pulsed mode. In some embodiments, Q-switching element 250 may be a passive Q switching element, or alternatively, an active Q-switching element.

In some embodiments, active Q-switching element 250 may be optionally an optical modulation unit, optionally positioned within a resonator. Optionally, an acousto-optic modulator (AOM), an electro-optic modulator (EOM), or an acousto-optic tunable filter (AOTF) may be included as an optical modulator in the optical modulation unit.

Optionally, laser system 200 may have a Q-switching element such as acousto-optic modulator (AOM) 250. In some embodiments, AOM 250 may be positioned in a light-path of the laser beam e.g., proximately along the longitudinal axis of the laser system 200. In one exemplary configuration, AOM 250 may be positioned at the second end of laser cavity 227, between the etalon 240B and output coupler (OC) 260. In other exemplary configuration, AOM 250 may be positioned between the gain medium 235 and the etalon 240A. Optionally, the length of the laser cavity 227 may be within a range of 1-500 mm, e.g., about 100 to 250 mm.

In some embodiments, AOM 250 may be configured to receive and modulate a seed laser beam. Optionally, the laser beam may be arranged to be generally incident at the Bragg angle to AOM 250. In some embodiments, AOM 250 may allow to produce a pulsed output beam. In some embodiments, AOM 250 may control the timing of the release of the pulse from the seed laser.

In another configuration, laser system 200 comprises a passive Q-switching element 250 instead of AOM 250. In some embodiments, passive Q-switching element may be configured to provide passive pulse switching of the laser beam. In some embodiments, a non-limiting example of a passive Q-switching element 250 is a saturable absorber (SA). In some embodiments, as laser system 200 provides passive Q-switching element 250 with a laser a short pulse laser beam may be produced. In some embodiments, non-limiting exemplary q-switching element 250 may comprise a material selected from doped ZnS crystals, and doped ZnSe crystals e.g., chromium doped ZnSe crystals, chromium doped ZnS crystals, Ho doped materials, such as, Ho:YAG, Ho:YLF, Ho:YAP Ho doped fiber or a combination thereof.

Optionally, SA comprises a semiconductor. Optionally, SA comprises a quantum dot. Optionally, SA comprises a doped crystal. In some embodiments, non-limiting exemplary doped crystals are selected from: chromium (II) doped zinc selenide (Cr:ZnSe) and chromium (II) doped zinc sulfide (Cr:ZnS). Optionally, the w/w (weight per weight) concentration of Cr²⁺ dopant in a doped crystal is between about 1% to about 20%, or optionally from 9 to 13%. In some cases, Cr:ZnSe and Cr:ZnS SA may have a relatively high absorption cross-sections, thus not requiring a focusing mode to a small area on the SA. In some embodiments, this may provide more flexibility with respect of the resonator. Optionally, the Cr:ZnSe and the Cr:ZnS SA have a low saturable intensity, which may lead to reduced risk of damage during Q-switched operation. Optionally, the Cr:ZnS crystal SA may be applied in several passive Q-switch (“PQS”) lasers, e.g. Ho:YAG, Tm:KY(WO₄), Tm:KLu(WO₄), or the like.

In some embodiments, laser system 200 may have an output coupler (OC) 260. In some embodiments, OC 260 may be positioned at the second end of laser cavity 227.

Optionally, OC may be positioned in a light-path of the laser beam. In some embodiments, OC 260 may transmit a portion of the optical power in the intracavity beam 270 outside the laser cavity to form the output beam. In some embodiments, OC 260 may be a component of an optical resonator that allows the extraction of a portion of the light from the laser's intracavity beam. In some embodiments, OC 260 may have a partially reflective (PR) coating, allowing a certain portion of the intracavity beam to transmit through. In some embodiments, OC may have a PR coating for a wavelength in the range of 1800-2200 nm, 1800-2000 nm, 2000-2200 nm and any range and value herein between. Optionally, PR coating has a reflectance in the range of 50-90%.

Optionally, the gain medium 235, the pump source 210, the input mirror 230, the output coupler 260, the first etalon 240A, the second etalon 240B, and the q-switching element 250 are at a light-pass of the laser beam.

Optionally, OC may be a plano-concave mirror or plano-convex mirror.

Optionally, the curvature radius of the plano-concave mirror may range from 100 to 400 mm, from 150 to 250 mm.

Optionally, laser system 200 may have a housing (not illustrated). In some embodiments, the housing may be made of a rigid, durable material, such as, without limitation, aluminum, stainless steel, a hard polymer and/or the like. In some embodiments, the housing may have a cylindrical, conical, rectangular or any other suitable shape. In some embodiments, the housing may prevent unwanted foreign elements from entering thereto.

In some embodiments, laser system 200 may further comprise controller 205 associated with laser system 200. In some embodiments, controller 205 may further be configured to: receive an input containing a desired depth and diameter tissue damage from a user interface, determine a frequency of a laser beam based on the desired diameter, determine a lasing power of the laser beam and number of pulses based on the desired depth, and control the laser system (e.g., laser system 200) to produce the laser beam. In some embodiments, controller 205 may receive a signal, containing a desired depth and diameter tissue damage, from an external device associated with laser system 200. In some embodiments, controller 205 may control a frequency and number of pulses and lasing power of laser system 200 to produce a desired ablation depth and coagulation diameter according to the received input. In some embodiments, a selected operating frequency and lasing power may be determined from a reference table, discussed hereinbelow under “Experimental Results”. In some embodiments, controller 105 may control pump 210 to produce a laser beam at a determined lasing power according to the received input. In some embodiments, controller 205 may control an active q-switching element 240 to produce a pulsed laser beam at a determined frequency according to the received input. In some embodiments the controller 205 control a scanning element. In some embodiment the controller 205 control the pulse frequency with the scanning elements, deliver to the same ablation well only fraction of the laser system 200 pulses. In some embodiment the controller 205 control the scanning element to create some ablation well at the same time, deliver each pulse to the next well, and repeating the pattern at the desired frequency.

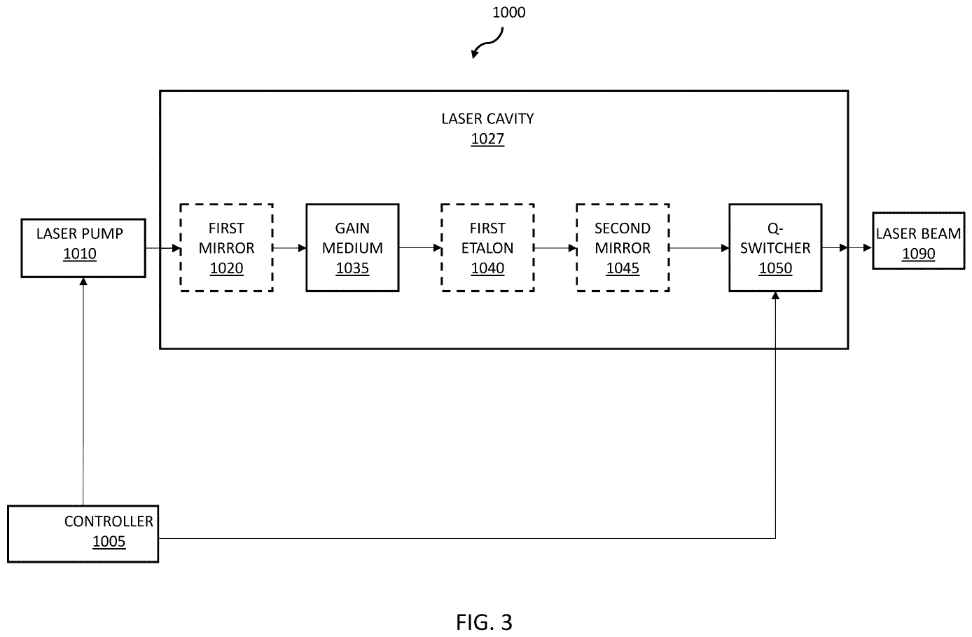

Reference is now made to FIG. 3 detailing a block diagram of a laser system according to some embodiments of the invention. In some embodiments, a laser system 1000 may comprise substantially the same components, elements, and units as laser system 100 or laser system 200 discussed hereinabove with respect to FIGS. 1 and 2. In some embodiments, a laser system 1000 may comprise a controller 1005 configured to perform substantially the same actions, controls, and decisions as controller 105 or 205 of laser systems 100 or 200 respectively, discussed hereinabove with respect to FIGS. 1 and 2.

In some embodiments, controller 1005 may be configured to control an active q-switching element 1050 as illustrated. In some embodiments, controlling an active q-switching element 1050 may comprise controlling a pulsed output beam of laser beam 1090, discussed hereinabove with respect to AOM 250 of laser system 200.

In some embodiments, laser system 1000 comprises a laser pump 1010 configured to optically pump a lasing medium. In some embodiments, laser pump 1010 may be controlled by controller 1005 or any suitable controller thereof. In some embodiments, laser pump 1010 may be controlled to operate in continuous-wave mode or quasi-continuous-wave mode or pulse mode. In some embodiments, laser pump 1010 may provide a laser beam to a first end of a laser cavity 1027.

In some embodiments, laser pump 1010 may provide a pump beam to a gain medium 1035. In some embodiments, gain medium 1035 comprises substantially the same capabilities, aspects, or embodiments as gain mediums 135 or 235 of laser systems 100 or 200, respectively. In some embodiments, gain medium 1035 may be disposed within a laser cavity 1027.

In some embodiments, q-switching element 1050 comprises substantially the same capabilities, aspects, or embodiments as q-switching elements 140 or 250 of laser systems 100 or 200, respectively. In some embodiments, q-switching element 1050 may be disposed within a laser cavity 1027.

In some embodiments, laser cavity 1027 may further comprise: a first mirror 1020, first etalon 1040, and second mirror 1045. In some embodiments, first mirror 1020 comprises substantially the same capabilities, aspects, or embodiments as first optical element 130 or 230 of laser systems 100 or 200, respectively. In some embodiments, second mirror 1045 comprises substantially the same capabilities, aspects, or embodiments as second optical element 145 or output coupler 260 of laser systems 100 or 200, respectively.

In some embodiments, first etalon 1040 comprises substantially the same capabilities, aspects, or embodiments as first etalon 240A of laser system 200. In some embodiments, first etalon 1040 is optionally paired with a second etalon (not illustrated) further disposed within laser cavity 1027. In some embodiments, at least one first or second etalon may be configured to provide tunability of laser beam 1090 with respect to a spectral range of laser beam 1090, as discussed hereinabove.

In some embodiments, laser beam 1090 may be emitted at a second end of laser cavity 1027. In some embodiments, laser beam 1090 may be a pulsed laser according to some aspects of the invention. In some embodiments, laser beam 1090 may be characterized by a pulse energy and frequency and laser wavelength as a result of controlling laser pump 1010 or first etalon 1040 as discussed hereinabove.

Reference is now made to FIG. 4, which is a block diagram depicting a computing device, which may be included within an embodiment of laser systems 100, 200, or 1000, according to some embodiments of the present invention. In some embodiments, computing device 1 is an embodiment of controller 105, 205 or 1005, configured to perform the same operations as discussed herein above. In some embodiments, computing device 1 may be any suitable controller, configured to control laser systems 100, 200, or 1000 according to some aspects of the invention.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. Although, for the sake of clarity, a single item of executable code 5 is shown in FIG. 4, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data related to correlations (e.g., a lookup table) between a frequency of a laser beam and a diameter of the tissue ablation, and a lasing power of the laser beam based on an ablation depth may be stored in storage system 6 and may be loaded from storage system 6 into memory 4 where it may be processed by processor or controller 2. In some embodiments, some of the components shown in FIG. 4 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

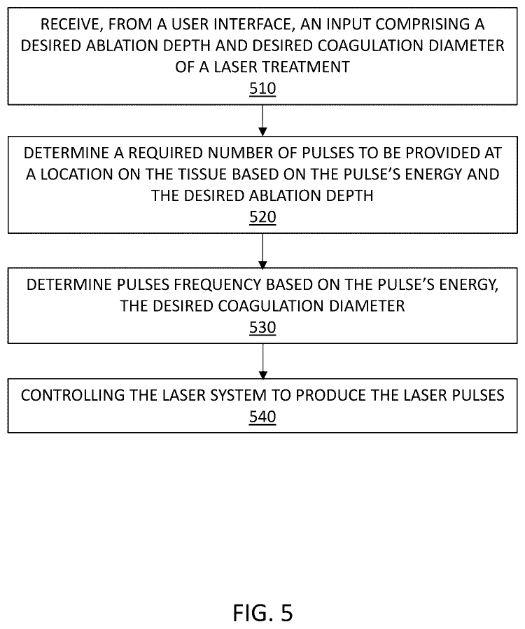

Reference is now made to FIG. 5, which is a flowchart of a method of controlling a laser system according to some aspects of the present invention. In some embodiments, steps 505 to 530 may be used to control laser system 100, 200, or 1000. In some embodiments, steps 505 to 530 may be controlled by controller 105, 205, or 1005 of laser systems 100, 200, or 1000, respectively, or any other suitable controller.

In step 510, an input may be received from a user interface, by controller thereof, comprising a desired ablation depth and desired coagulation diameter of a laser treatment. In some embodiments, the user interface may be associated or included in controller 105, 205, or 1005, or may be in communication with any one of controller 105, 205, or 1005.

In step 520, a required number of pulses to be provided at a location on the tissue and may be determined based on the pulse's energy and the desired ablation depth. For example, a look up table or look up tables comprising the required number of pulses associated with the desired ablation depth may be stored in a memory, such as, storage system 6. Nonlimiting examples for such a table is given and discussed below with respect to Table 1. As the amount of energy provided by each pulse is known, e.g., 1 mJ, 1.2 mJ, 2 mJ, 3 mJ, 5 mJ and the like, the controller may calculate the number of required pulses. In the nonlimiting example of Table 1, in order to ablate to a depth of 1 mm, 100 mJ are required. Accordingly, if each laser pulse provides 4 mJ, 25 pulses will be required to ablate a depth of 1 mm.

In step 530, a required pulses frequency may be determined based on a pulse's energy and the desired coagulation diameter. For example, a look up table or look up tables comprising the frequency desired coagulation diameter may be stored in a memory, such as, storage system 6. Nonlimiting examples for such tables are given and discussed below with respect to Table 2. In the nonlimiting example of Table 2, if a coagulation diameter of 150 μm is required the pluses are provided at 10 Hz.

In step 540, a laser system or controller thereof may control the laser system to produce the laser pulse, according to the determined frequency and number of pulses.

In some embodiments, the laser system may be controlled to emit a pulsed laser, wherein a duration of the pulse is between 2 to 100 nanoseconds, 2 to 20 nanoseconds, 20 to 50 nanoseconds, 50 to 100 nanoseconds and any range and value herein between. In some embodiments, the laser beam produced may be characterized by a wavelength of 2 μm regime. In some embodiments, the laser beam may be characterized by energy (also referred to as “lasing power”) of at least 1 mJ, 1 to 15 mJ, 1 to 5 mJ, 5 to 15 mJ and any range and value herein between.

In some embodiments, an electrical power may be supplied to the laser system, in order to produce said laser beam, wherein said electrical power may be between 1 to 50 Watts, 1 to 16 Watts, 10 to 30 Watts, 25 to 50 Watts and any range and value herein between.

In some embodiments, the laser beam may be focused into a Raman gain crystal. In some embodiments, the laser system may emit a spot diameter size of a laser beam between 20 to 200 microns, 20 to 40 microns, 40 to 100 microns, 100 to 200 microns and any range and value herein between.

In some embodiments, in the case of active q-switching, a q-switching element 140, 250, or 1050 of laser systems 100, 200, or 1000 may be controlled to produce a pulsed laser output, as discussed hereinabove with respect to AOM 250 of laser system 200.

### Experimental Results

In some embodiments, a laser device as described herein above may control an energy (also referred to as “lasing power” hereinabove) and frequency output based on a desired ablation depth and diameter input. Table 1 herein below details an exemplary resulting depth output of a laser beam, according to some embodiments of the invention, based on an energy input of the device:

TABLE 1

| Energy (mJ) | Depth (mm) |
|---|---|
| 20 | 0.45-0.55 |
| 40 | 0.75-0.85 |
| 100 | 0.95-1 |
| 200 | 1.2 |

Table 2 herein below details an exemplary resulting diameter (width) output of a laser beam, according to some embodiments of the invention, based on a frequency input of the device:

TABLE 2

| Pulses frequency (Hz) | Width (μm) |
|---|---|
| 10 | 150 |
| 25 | 200 |
| 800 | 500 |

In some embodiments, to produce a desired ablation, a combination of pulse frequencies and lasing energy (therefore the number of pulses) may result in a desired ablation depth and coagulation diameter. In a non-limiting combination, for example, a desired ablation with a depth of 1 mm and coagulation diameter of 150 μm may be produced by operating a laser at a frequency of 10 Hz with and 10 pulses of 10 mJ, to produce a total lasing energy of 100 mJ.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments have been presented. Each of these embodiments may of course include features from other embodiments presented, and embodiments not specifically described may include various features described herein.

1. A laser system, comprising:
a laser cavity;
a gain medium disposed within the laser cavity configured to produce a laser pulses at a specific wavelength;
a pump configured to optically pump a lasing medium; and
a controller, configured to:
receive, from a user interface, an input comprising a desired ablation depth and desired coagulation diameter of a laser treatment;
determine a required number of pulses to be provided at a location on the tissue based on the pulse's energy and the desired ablation depth, and
determine pulses frequency based on the pulse's energy, the desired coagulation diameter, and
control the laser system to produce the laser pulses.

2. The laser system of claim 1, further comprising a q-switching element positioned within the laser cavity configured to produced pulsating laser.

3. The laser system of claim 1, wherein the laser pulses have a wavelength between 1850-2200 nm.

4. The laser system of claim 1, wherein the laser pulses have pulse energy between 0.5-15 mJ.

5. The laser system of claim 1, wherein the laser pulses have a pulse duration between 2-150 ns.

6. The laser system of claim 1, wherein the controller is configured to determine the frequency and the required number of pulses based on data stored in a storage system associated with the controller.

7. The laser system of claim 6, wherein the data is stored in a lookup table.

8. The laser system of claim 1, wherein the required number of pulses is determined based on the desired depth of the ablation.

9. The laser system of claim 1, wherein the required frequency is determined based on the desired diameter of the ablation.

10. The laser system of claim 9, wherein the desired diameter of the ablation is between 10 to 150 μm.

11. The laser system of claim 2, wherein the q-switching element is selected from an active q-switching element and a passive q-switching element.

12. The laser system of claim 11, wherein the passive q-switching element comprises a saturable absorber configured to provide passive pulse switching of a laser beam.

13. The laser system of claim 12, wherein the saturable absorber comprises Cr:ZnS or Cr:ZnSe.

14. The laser system of claim 11, wherein the active q-switching element comprises an Acousto-Optic-Modulator (AOM) configured to provide active pulse switching of a laser beam.

15.-21. (canceled)

22. The laser system of claim 1, wherein the laser beam is capable of tuning to a spectral range of at least 10 nm.

23. The laser system of claim 1, wherein the laser beam is characterized by a wavelength of Infrared (IR) spectrum.

24. The laser system of claim 1, wherein the laser beam is characterized by a wavelength between 1850 to 2020 nm.

25. The laser system of claim 1, wherein the gain medium comprises a crystal selected from a group consisting of: Yttrium Aluminum Garnet (YAG), Yttrium Aluminum Phosphorus (YAP), and Yttrium Lithium Fluoride (YLF).

26. A method for controlling a laser system, the method comprising:
receiving, from a user interface, an input comprising a desired ablation depth and desired coagulation diameter of a laser treatment;
determining a required number of pulses to be provided at a location on the tissue on the pulse's energy and the desired ablation depth;
determining the pulses' frequency based on the pulse's energy, the desired coagulation diameter, and
controlling the laser system to produce the laser pulses.

27.-34. (canceled)
